(19) United States
(12) Patent Application Publication
(10) Pub. No.: US 2010/0010609 A1
(43) Pub. Date: Jan. 14, 2010

(54) Multi-Channel Electrode for Cochlear Implants Having a Plurality of Contacts Distributed Over the Length of the Electrode

(75) Inventors: Sasan J. Hamzavi, Vienna (AT); Christoph Arnoldner, Vienna (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/438,630

(22) PCT Filed: Aug. 24, 2007
(86) PCT No.: PCT/AT2007/000405

§ 371 Date: May 21, 2009

(30) Foreign Application Priority Data
Aug. 24, 2006 (AT) A 1415/2006

(51) Int. Cl. A61F 11/04; A61N 1/05

(52) U.S. Cl. 607/137

(57) ABSTRACT

In a multi-channel electrode for cochlear implants, having a plurality of contacts (2, 4, 5, 6) distributed over the length of the electrode, the distance between the active contacts is selected to be greater in an apical end region than in a basal region.

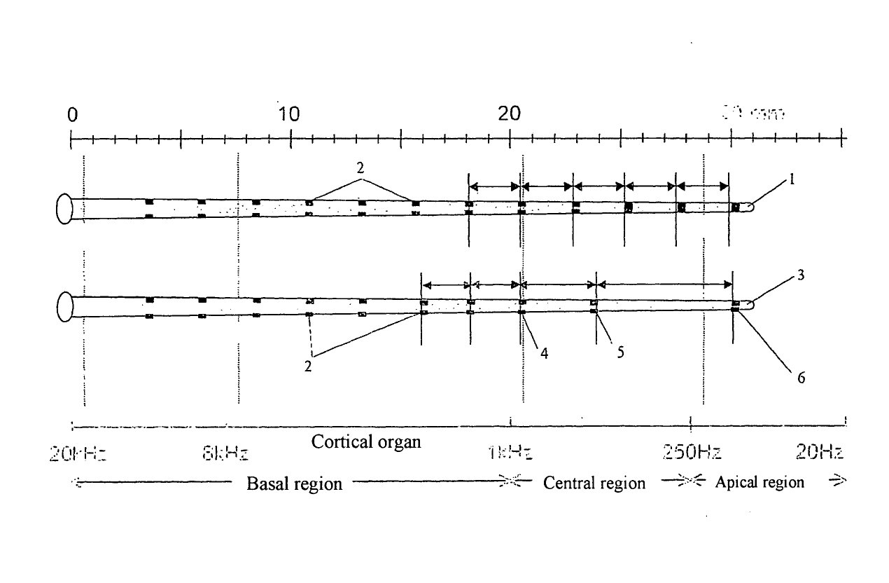

The invention relates to a multi-channel electrode for cochlear implants, having a plurality of contacts distributed over the length of the electrode.

Cochlear implants are used to transform electrical signals generated by microphones so that, as the result of electrodes introduced into the cochlea, corresponding nerve stimulation is made possible which allows the stimulus to be correspondingly transmitted to the hearing center of the brain. Such cochlear implants thus avoid the mechanical transmission of sound to the neuronal system, and allow nerves or nerve endings to be directly stimulated electrically, thereby substituting for the mechanical stimulus, functioning in the healthy ear, for triggering stimulus signals. This requires a high level of electronic complexity and use of a complex algorithm, whereby the electronically generated signals are sent to multi-channel electrodes, and the signals as a rule are cyclically fed to the individual channels of the multi-channel electrode.

For known cochlear implants, the electrodes themselves are inserted into the cochlea, a given insertion depth being achievable depending on the design of the electrode. Conventional multi-channel electrodes have 12 or more contacts, such electrodes being insertable only to an insertion depth of typically 17 to 25 mm. The cochlea represents an organ which develops very early in the human body; its absolute size in a newborn infant is not significantly different from that in an adult. In principle, cochlear implants may be successfully inserted in hearing-impaired newborn infants, who by use of a cochlear implant may learn to talk, or for adult persons for whom, after learning speech, after a given period in which the hearing functioned properly a mechanical defect has appeared which reduces or eliminates the ability to hear. In these cases the known electronic algorithms may be used to provide a substitute electrical stimulation, resulting in the ability to comprehend speech.

In the literature, the limitation of the insertion depth has generally been based on the fact that only in the basal and central regions does the cochlea have sufficient nerve endings which, when stimulated, are able to provide meaningful evaluation in the hearing center. For this reason the insertion depth of the electrodes has been limited to the basal and central regions; longer electrodes have infrequently appeared on the market which in principle would also be able to penetrate into the apical region of the cortical organ. Association of the electrode position with the relative location inside the cortical organ has been attempted, resulting in the finding that the basal region is particularly suited for receiving frequencies from 1 kHz to 8-10 kHz, whereas the subsequent interior central region preferentially detects the range of 1 kHz and lower. Regions located farther inward are thus able to detect even lower frequencies.

Known multi-channel electrodes for cochlear implants are characterized by a maximum length of 32 mm, the distance between the electrodes being held constant over the insertion depth.

A conventional multi-channel electrode is known from U.S. Pat. No. 7,076,308 B1, which describes an improved method for determining and adjusting the particular stimulation currents required.

U.S. Pat. No. 7,039,466 B1 describes a cochlear implant in which the apical region of the cochlea is stimulated at a decreased stimulation rate compared to the basal region.

A multi-channel electrode for a cochlear implant is also known from US 2006/136030 A1.

The object of the present invention is to provide a multi-channel electrode of the type stated at the outset, by means of which not only acoustic perception, but in particular also speech comprehension is further improved, and undesired interferences resulting from crosstalk from adjacent channels are further inhibited. This object is achieved essentially by the fact that in the aforementioned multi-channel electrode according to the invention, the distance between the active contacts is selected to be greater in an apical end region than in a basal region. Surprisingly, for such a design it has been shown that in the apical end region not only is there better detection of low frequencies, for example, but also speech comprehension is improved for correspondingly large distances between active contacts in the apical end region, and thus at an insertion depth which is generally greater than that for known multi-channel electrodes. This is all the more surprising since in the literature the apical end inside the cochlea has been described as being less sensitive, so that low frequencies do not necessarily improve speech comprehension. Increasing the distance between the active contacts in the apical region, however, has resulted in the unexpected finding that with a relatively low overall number of channels an increase in the level of speech comprehension may be achieved.

The design according to the invention is advantageously such that the region with the larger contact spacing corresponds to an insertion depth of the electrode of greater than 20 mm, preferably 20 to 32 mm, thereby ensuring that the multi-channel electrode can actually be inserted at its apical end region all the way to the end of the cochlea. The design may preferably be such that the contact spacing is increased in at least two spacing levels, whereby in the first subregion corresponding to a central region of the electrode the contact spacing is increased by 25 to 50%, in particular by approximately ⅓, and in the apical end corresponds to 1.5 to 2.5 times, in particular 2 times, of the previously increased contact spacing, the distance between the contacts in the apical region preferably corresponding to at least 2.5 times the distance between the contacts in the basal region of the electrode. Such a design results in a particularly high level of speech comprehension, while at the same time the number of contacts is relatively low. The test electrodes have 12 or fewer contacts, whereas customary electrodes of known design, which achieve insertion depths of 16 to 22 mm, have a far greater number of contacts. At the same time, the reduction in the number of contacts results in a further decrease in channel crosstalk.

A particularly advantageous embodiment with particularly good speech comprehension may be achieved using a design in which the contact spacings from the basal end to the apical end in the transition region between the basal and the central regions initially decrease, and in the central and apical regions increase.

The invention is explained in greater detail with reference to designs of a standard electrode and an electrode modified according to the invention illustrated in the drawing. In the drawing, reference numeral 1 denotes a standard electrode having 12 contacts, the distance between the contacts being held essentially constant. The contacts are denoted by reference numeral 2, and are individually connected to the associated electronic control system via separate lines. The electrode according to the invention denoted by reference numeral 3 has essentially constant contact spacings in the basal region. This region of the cortical organ is usually responsible for the detection of frequencies between 15 kHz and 1 kHz. In the subsequent central region corresponding to an insertion depth of greater than 20 mm, the corresponding contact spacing is increased by a factor of 1.33, the corresponding contacts then being denoted by reference numerals 4 and 5. The distance between contact 5 and contact 6 in the apical end of the electrode 3 then corresponds to approximately twice the distance between contacts 4 and 5, and extends far into the apical region, thus being situated at an insertion depth of approximately 30 mm. In the drawing, the particular corresponding insertion is plotted at the upper edge. The corresponding frequency sensitivity of the cortical organ and the individual regions, namely, the basal, central, and apical regions corresponding to the particular insertion depth, are illustrated at the lower edge of the drawing.

1-5. (canceled)

6. A cochlear implant electrode comprising:
a multi-channel cochlear implant electrode having a defined length between a basal region and an apical end; and
a plurality of electrode contacts distributed over the electrode length, wherein the distance between electrode contacts towards the apical end is greater than the distance between electrode contacts towards the basal region.

7. A cochlear implant electrode according to claim 1, wherein electrode contacts towards the apical end correspond to an electrode insertion depth of greater than 20 mm.

8. A cochlear implant electrode according to claim 7, wherein the electrode insertion depth is in between 20 mm and 32 mm.

9. A cochlear implant electrode according to claim 1, wherein the distance between electrode contacts in a middle region of electrode contacts is greater the distance between electrode contacts towards the basal region and less than the distance between electrode contacts towards the apical end.

10. A cochlear implant electrode according to claim 1, wherein the distance between the electrode contacts towards the apical end is at least 2.5 times the distance between the electrode contacts towards the basal region.
